(19) United States
(12) Patent Application Publication
(10) Pub. No.: US 2025/0171711 A1
(43) Pub. Date: May 29, 2025

(54) PLANT AND PROCESS FOR FRACTIONATING FATTY ACID MIXTURES

(71) Applicant: SULZER MANAGEMENT AG, Winterthur (CH)

(72) Inventors: Wan Seang KEK, Shanghai (CN); Keen Hoe LUI, Singapore (SG); Quan YANG, Singapore (SG); Patrick FARQUET, Winkel (CH)

(21) Appl. No.: 18/725,730

(22) PCT Filed: Jan. 16, 2023
(86) PCT No.: PCT/EP2023/050884

§ 371 Date: Jun. 28, 2024

(30) Foreign Application Priority Data
Jan. 17, 2022 (EP) 22151845.9

(51) Int. Cl. C11C 1/10; C11C 1/00

(52) U.S. Cl. CPC C11C 1/10; C11C 1/005

(57) ABSTRACT

A method for fractionating a crude composition comprising at least six fatty acids each having a different chain length into at least four fractions is provided. The method comprises feeding the crude composition into a first distillation column to obtain a first overhead fraction and a first bottom fraction, feeding the first bottom fraction to a second distillation column to obtain a second overhead fraction and a second bottom fraction, feeding the second bottom fraction to a third distillation column to obtain a third overhead fraction and a third bottom fraction, and feeding the third bottom fraction to a fourth distillation column to obtain a fourth overhead fraction, a fourth side fraction and a fourth bottom fraction. At least one of the first distillation column, the second distillation column and the third distillation column is a middle dividing-wall column, from which also at least two side fractions are obtained.

### CROSS-REFERENCE TO RELATED APPLICATION

This application is a U.S. National stage application of International Application No. PCT/EP2023/050884, filed on Jan. 16, 2023. This U.S. National stage application claims priority under 35 U.S.C. § 119 (a) to European Patent Application No. 22151845.9, filed in Europe on Jan. 17, 2022, the entire contents of which are hereby incorporated herein by reference.

### BACKGROUND

### Technical Field

The present disclosure relates to a plant and process for fractionating fatty acid mixtures, such as in particular for fractionating palm kernel fatty acid or coconut fatty acid.

### Background Information

A plurality of natural occurring fatty acid mixtures, such as palm kernel fatty acid and coconut fatty acid, are complex mixtures comprising several fatty acids having different chain lengths. For instance, palm kernel fatty acid and coconut fatty acid comprise several even-chained fatty acids, i.e. monocarboxylic acids, namely C₆-, C₈-, C₁₀-, C₁₂-, C₁₄-, C₁₆-, C₁₈- and longer fatty acids. More specifically, palm kernel fatty acid comprises 0.3% by weight of C₆-fatty acids, 4.4% by weight of C₈-fatty acids, 3.7% by weight of C₁₀-fatty acids, 48.2% by weight of C₁₂-fatty acids, 15.6% by weight of C₁₄-fatty acids, 7.8% by weight of C₁₆-fatty acids, 19.8% by weight of C₁₈-fatty acids and 0.2 of other compounds, such as non-fatty acid impurities, for instance ketones, alkenes or the like. The C₁₈-fatty acid fraction comprises the saturated C₁₈-stearic acid, the mono-unsaturated C₁₈-oleic acid and the di-unsaturated C₁₈-linoleic acid. In order to obtain the single fatty acids from these mixtures, the respective fatty acid mixtures must be fractionated.

This fractionation is usually performed by distillation, wherein 7 distillation columns are needed to obtain 7 fractions from palm kernel fatty acid, namely the C₆-, C₈-, C₁₀-, C₁₂-, C₁₄-, C₁₆- and C₁₈ₚₗᵤₛ-fatty acid fractions. More specifically, the palm kernel fatty acid mixture is distilled in a first distillation column so as to obtain as overhead fraction a rather impure C₆-fatty acid fraction, as side fraction a C₈₋₁₀-fatty acid fraction and a bottom fraction with the remaining fatty acids. The C₆- and C₈₋₁₀-fatty acid fractions are further purified in two (i.e. the second and third) distillation columns which are erected under campaign running mode, to a rather pure C₆-fatty acid fraction, a rather pure C₈-fatty acid fraction and a rather pure C₁₀-fatty acid fraction. The bottom fraction of the first distillation column is distilled in a fourth distillation column so as to obtain as side fraction a C₁₂-fatty acid fraction and a bottom fraction with the remaining fatty acids, wherein the bottom fraction of the fourth distillation column is distilled in a fifth distillation column so as to obtain as side fraction a C₁₄-fatty acid fraction and a bottom fraction with the remaining fatty acids, wherein the bottom fraction of the fifth distillation column is distilled in a sixth distillation column so as to obtain as side fraction a C₁₆-fatty acid fraction and a bottom fraction with the remaining fatty acids, and wherein the bottom fraction of the sixth distillation column is distilled in a seventh distillation column so as to obtain as side fraction a C₁₈ₚₗᵤₛ-fatty acid fraction. Due to the seven distillation columns, the prior art method has a comparably high energy consumption. Moreover, the method is not continuous on account of the campaign mode. In addition, a plant with seven distillation columns requires a large plot area.

It has been already proposed to perform such a fractionation in six distillation columns connected in series with each other. In the first distillation column, a C₆-fatty acid side fraction and another C₈-fatty acid side fraction are obtained, whereas the bottom product is sent to a second distillation column in which a C₁₀-fatty acid side fraction is obtained. The bottom product of the second distillation column is sent to a third distillation column in which a C₁₂-fatty acid side fraction is obtained, whereas the bottom fraction of the third distillation column is sent to a fourth distillation column in which a C₁₄-fatty acid side fraction is obtained, the bottom fraction of the fourth distillation column is sent to a fifth distillation column in which a C₁₆-fatty acid side fraction is obtained and the bottom fraction of the fifth distillation column is sent to a sixth distillation column in which a C₁₈ₚₗᵤₛ-fatty acid side fraction is obtained. However, this method also has a comparably high energy consumption and requires a comparably large plot area. Moreover, the purities of the single fractions are less than those of the fractions obtained with the aforementioned seven columns-method.

In view of all this, the object underlying the present disclosure is to provide a method and a plant for fractionating fatty acid mixtures, in particular palm kernel fatty acid or coconut fatty acid, which is continuous, which leads to fractions with a high purity, which only requires a comparably small plot area and which has, relative to the obtained purity, a comparably low energy consumption.

### SUMMARY

In accordance with the present disclosure, this object is satisfied by providing a method for fractionating a crude composition comprising at least six fatty acids having each a different chain length into at least four fractions, the method comprising:

feeding the crude composition into a first distillation column and distilling it so as to obtain at least a first overhead fraction and a first bottom fraction,
feeding the first bottom fraction of the first distillation column to a second distillation column and distilling it so as to obtain at least a second overhead fraction and a second bottom fraction,
feeding the second bottom fraction of the second distillation column to a third distillation column and distilling it so as to obtain at least a third overhead fraction and a third bottom fraction, and
feeding the third bottom fraction of the third distillation column to a fourth distillation column and distilling it so as to obtain a fourth overhead fraction, at least one side fraction and a fourth bottom fraction,

wherein at least one of the first distillation column, the second distillation column and the third distillation column is a middle dividing-wall column, from which at least two side fractions are obtained.

This solution is based on the finding that by replacing one or two or even three distillation columns of the conventional methods by one or two or even three middle dividing-wall distillation columns, from at least one of which and preferably from each of which at least two side fractions are obtained, not only the energy consumption can be significantly decreased in a continuous process, namely—depending on the specific embodiment by 10 to 25%—, but also the purity of the obtained fatty acid fractions can be improved. In particular, at least one of the at least two side fractions obtained in the middle dividing-wall column may be a fatty acid product fraction, whereas the other is a purge fraction allowing the quality of the fatty acid product fraction to be increased by purging out the impurities existing in the crude composition in particular between even-numbered fatty acids. More specifically, the method in accordance with the present disclosure allows for the production of C₆-, C₈-, C₁₀-, C₁₂-, C₁₄ -and C₁₆-fractions having a purity of at least 99.5% each and a C₁₈ₚₗᵤₛ-fraction containing unsaturated, monounsaturated and di-unsaturated C₁₈-fatty acids (i.e. C_{18:0}-, C_{18:1}- and C_{18:2}-fatty acids) and fatty acids having a longer chain length having a purity of at least 99.0%, and this, as set out above, with a reduced energy consumption. In addition, because of the reduced number of distillation columns, the required plot area for the plant is reduced. Moreover, the total residence time is excellently short and the yield is excellently high.

The numeration of the distillation columns is independent from the nature of the distillation columns. Thus, if there is a series of a non-dividing-wall distillation column, a middle dividing-wall distillation column, a further non-dividing-wall distillation column and a further middle dividing-wall distillation column these are numerated as first non-dividing-wall distillation column, as second middle dividing-wall distillation column, as third non-dividing-wall distillation column and as fourth middle dividing-wall distillation column.

According to a first preferred embodiment of the present disclosure, the method comprises the use of one middle-wall dividing-wall column. The middle-wall dividing-wall column is preferably any of the first distillation column, the second distillation column and the third distillation column. It is more preferred in this embodiment that the middle-wall dividing-wall column is the first or second distillation column.

According to a first variant of an embodiment of the present disclosure, the method comprises:

feeding the crude composition into a first non-dividing-wall distillation column and distilling it so as to obtain at least an overhead fraction, a side fraction and a bottom fraction,
feeding the bottom fraction of the first non-dividing-wall distillation column to a second middle dividing-wall distillation column and distilling it so as to obtain at least an overhead fraction, at least two side fractions and a bottom fraction,
feeding the bottom fraction of the second middle dividing-wall distillation column to a third non-dividing-wall distillation column and distilling it so as to obtain at least an overhead fraction, a side fraction and a bottom fraction, and
feeding the bottom fraction of the third non-dividing-wall distillation column to a fourth non-dividing-wall distillation column and distilling it so as to obtain at least an overhead fraction, a side fraction and a bottom fraction.

According to a second variant of an embodiment of the present disclosure, the method comprises:

feeding the crude composition into a first middle dividing-wall distillation column and distilling it so as to obtain at least an overhead fraction, at least two side fractions and a bottom fraction,
feeding the bottom fraction of the first middle dividing-wall distillation column to a second non-dividing-wall distillation column and distilling it so as to obtain at least an overhead fraction, a side fraction and a bottom fraction.
feeding the bottom fraction of the second non-dividing-wall distillation column to a third non-dividing-wall distillation column and distilling it so as to obtain at least an overhead fraction, a side fraction and a bottom fraction, and
feeding the bottom fraction of the third non-dividing-wall distillation column to a fourth non-dividing-wall distillation column and distilling it so as to obtain at least an overhead fraction, a side fraction and a bottom fraction.

The method of the first preferred embodiment of the present disclosure allows fractionation of a fatty acid mixture comprising at least six fatty acids having each a different chain length in particular into five fractions of which four fractions are very pure, such as to fractionate palm kernel fatty acid or coconut fatty acid into a very pure C₁₂-fatty acid fraction having a purity of at least 99.5% by weight, a very pure C₁₄-fatty acid fraction having a purity of at least 99.5% by weight, a very pure C₁₆-fatty acid fraction having a purity of at least 99.5% by weight, a very pure C₁₈ₚₗᵤₛ-fatty acid fraction having a purity of at least 99.0% by weight and a C₈₋₁₀-fatty acid fraction having a purity of at least 95.0% by weight.

According to a second preferred embodiment of the present disclosure, the method comprises the use of two middle-wall dividing-wall columns. Preferably, the two middle-wall dividing-wall columns are the second and third distillation columns.

According to a variant of an embodiment of the present disclosure, the method comprises:

feeding the crude composition into a first non-dividing-wall distillation column and distilling it so as to obtain at least an overhead fraction, a side fraction and a bottom fraction,
feeding the bottom fraction of the first non-dividing-wall distillation column to a second middle dividing-wall distillation column and distilling it so as to obtain at least an overhead fraction, at least two side fractions and a bottom fraction,
feeding the bottom fraction of the second middle dividing-wall distillation column to a third middle dividing-wall distillation column and distilling it so as to obtain at least an overhead fraction, at least two side fractions and a bottom fraction, and
feeding the bottom fraction of the third middle dividing-wall distillation column to a fourth non-dividing-wall distillation column and distilling it so as to obtain at least an overhead fraction, a side fraction and a bottom fraction.

The method of this second preferred embodiment allows fractionation of a fatty acid mixture comprising at least six fatty acids having each a different chain length in particular into seven fractions of which six fractions are very pure, such as to fractionate palm kernel fatty acid or coconut fatty acid into a very pure C₈-fatty acid fraction having a purity of at least 99.5% by weight, a very pure C₁₀-fatty acid fraction having a purity of at least 99.5% by weight, a very pure C₁₂-fatty acid fraction having a purity of at least 99.5% by weight, a very pure C₁₄-fatty acid fraction having a purity of at least 99.5% by weight, a very pure C₁₆-fatty acid fraction having a purity of at least 99.5% by weight, a very pure C₁₈ₚₗᵤₛ-fatty acid fraction having a purity of at least 99.0% by weight and a C₆-fatty acid fraction having a purity of at least 95.0% by weight.

In order to allow the purity of the seventh fraction to be increased and thus to allow fractionation of a fatty acid mixture into seven different fractions with each having an excellently high purity, it is suggested according to a third preferred embodiment of the present disclosure that the method comprises the use of three or even more middle-wall dividing-wall columns. Preferably, the method of this third embodiment comprises in addition to the steps of the second preferred embodiment the step of feeding the overhead fraction of the first distillation column to a fifth middle dividing-wall distillation column and distilling it so as to obtain an overhead fraction, at least two side fractions and a bottom fraction.

Thus, it is preferred in the third embodiment that the method comprises:

feeding the crude composition into a first non-dividing-wall distillation column and distilling it so as to obtain at least an overhead fraction and a bottom fraction.

feeding the bottom fraction of the first non-dividing-wall distillation column to a second middle dividing-wall distillation column and distilling it so as to obtain at least an overhead fraction. at least two side fractions and a bottom fraction.

feeding the bottom fraction of the second middle dividing-wall distillation column to a third middle dividing-wall distillation column and distilling it so as to obtain at least an overhead fraction, at least two side fractions and a bottom fraction,
feeding the bottom fraction of the third middle dividing-wall distillation column to a fourth non-dividing-wall distillation column and distilling it so as to obtain at least an overhead fraction, a side fraction and a bottom fraction, and
feeding the overhead fraction of the first distillation column to a fifth middle dividing-wall distillation column and distilling it so as to obtain an overhead fraction, at least two side fractions and a bottom fraction.

The third preferred embodiment allows fractionation of a fatty acid mixture in particular into seven fractions, with all seven fractions being very pure, such as to fractionate palm kernel fatty acid or coconut fatty acid into a very pure C₆-fatty acid fraction having a purity of at least 99.5% by weight, a very pure C₈-fatty acid fraction having a purity of at least 99.5% by weight, a very pure C₁₀-fatty acid fraction having a purity of at least 99.5% by weight, a very pure C₁₂-fatty acid fraction having a purity of at least 99.5% by weight, a very pure C₁₄-fatty acid fraction having a purity of at least 99.5% by weight, a very pure C₁₆-fatty acid fraction having a purity of at least 99.5% by weight and a very pure C₁₈ₚₗᵤₛ-fatty acid fraction having a purity of at least 99.0% by weight.

In a further embodiment of the present disclosure, it is proposed that from at least one and preferably from each of the middle dividing-wall columns at least three side fractions are obtained.

In accordance with an embodiment of the present disclosure, the method uses at least one, preferably at least two and more preferably at least three middle dividing-wall distillation columns. A middle dividing-wall distillation column is a distillation column, which comprises a wall partially separating or dividing, respectively, the middle portion of the distillation column into two parts, in particular into two halves so that the middle dividing-wall distillation column comprises above the dividing-wall a top section, below the dividing-wall a bottom section, on one side of the dividing-wall a first middle section and on the opposite side of the dividing-wall a second middle section. The middle dividing-wall distillation column comprises preferably one such dividing-wall, but not two or more thereof. More specifically, the dividing-wall extends at least essentially vertically and is located-seen in the vertical direction-in the middle of the distillation column so as to divide the middle portion of the distillation column into two parts, however leaving the uppermost and lowermost part of the distillation column undivided. Essentially vertically downwards means in this connection that the angle between the dividing-wall and the length axis of the first dividing-wall distillation column (which corresponds to the vertical direction) is at most 20°, preferably at most 10°, more preferably at most 5° and most preferably 0°.

It is preferred that the feed of at least one and preferably each of the middle dividing-wall distillation columns is fed into the first middle section of the middle dividing-wall distillation column, wherein from at least one and preferably each of the middle dividing-wall distillation columns at least one of the at least two side fractions is withdrawn from the top section and at least one of the at least two side fractions is withdrawn from the second middle section of the middle dividing-wall distillation column

In the embodiment, in which from at least one and preferably from each of the middle dividing-wall columns at least three side fractions are obtained, it is preferred that the feed of at least one and preferably each of the middle dividing-wall distillation columns is fed into the first middle section of the middle dividing-wall distillation column, wherein from at least one and preferably each of the middle dividing-wall distillation columns at least two of the at least three side fractions are withdrawn from the top section and at least one of the at least three side fractions is withdrawn from the second middle section of the middle dividing-wall distillation column.

Preferably, at least one and preferably each of the middle dividing-wall distillation columns comprise a dividing-wall extending, seen from the bottom to the top of the middle dividing-wall distillation column, from a point being located at 10 to 40% of the distance from the bottom to the top of the middle dividing-wall distillation column to a point being located at 50 to 90% of the distance from the bottom to the top of the middle dividing-wall distillation column. The dividing-wall is oriented at least essentially vertically downwards so that the middle dividing-wall distillation column comprises above the dividing-wall a top section functioning as refinement distillation section, below the dividing-wall a bottom section functioning as distillation section, on one side of the dividing-wall a first middle section functioning as feed section and on the opposite side of the dividing-wall a second middle section functioning as lateral extraction section.

More preferably, the dividing-wall of at least one of the middle dividing-wall distillation columns and preferably of all of the middle dividing-wall distillation columns extends, seen from the bottom to the top of the middle dividing-wall distillation column, from a point being located at 20 to 40% of the distance from the bottom to the top of the middle dividing-wall distillation column to a point being located at 50 to 80% of the distance from the bottom to the top of the middle dividing-wall distillation column. Most preferably, the dividing-wall of at least one of the middle dividing-wall distillation columns and preferably of all of the middle dividing-wall distillation columns extends, seen from the bottom to the top of the middle dividing-wall distillation column, from a point being located at 25 to 35% of the distance from the bottom to the top of the middle dividing-wall distillation column to a point being located at 55 to 75% of the distance from the bottom to the top of the middle dividing-wall distillation column.

The length of dividing-wall relative to the height of the middle dividing-wall distillation column is for at least one middle dividing-wall distillation column and preferably for all middle dividing-wall distillation columns 20 to 80%, i.e. in at least one and preferably each of the middle dividing-wall distillation columns the dividing-wall extends over 20 to 80% of the height of the middle dividing-wall distillation column. The height of the middle dividing-wall distillation column is defined in the present disclosure as the straight distance between the top and the bottom of the middle dividing-wall distillation column. More preferably, in at least one and preferably each of the middle dividing-wall distillation columns the dividing-wall extends over 30 to 70% of the height of the middle dividing-wall distillation column and most preferably, in at least one and preferably each of the middle dividing-wall distillation columns the dividing-wall extends over 30 to 60% of the height of the middle dividing-wall distillation column.

Good results are in particular obtained, when the method in accordance with the present disclosure is performed so that at least one and preferably each of the middle dividing-wall distillation columns is operated so that it has 25 to 60 theoretical stages.

In accordance with a further embodiment of the present disclosure, the method in accordance with the present disclosure is performed so that at least one and preferably each of the middle dividing-wall distillation columns is operated so that its top section comprises during the operation 5 to 15 theoretical stages, its middle section comprises 10 to 30 theoretical stages and its bottom section comprises 10 to 15 theoretical stages.

As set out above, it is preferred that the feed of at least one and preferably each of the middle dividing-wall distillation columns is fed into the first middle section of the respective middle dividing-wall distillation column.

It is further preferred that from at least one and preferably each of the middle dividing-wall distillation columns at least one of the at least two side fractions is withdrawn from the top section and at least one of the at least two side fractions is withdrawn from the second middle section of the respective middle dividing-wall distillation column.

In another embodiment of the present disclosure, it is proposed to withdraw during the method from at least one and preferably each of the middle dividing-wall distillation columns a purge in the top section of the middle dividing-wall distillation column. If at least three side fractions are obtained in the middle dividing-wall distillation column, at least one of the at least three side fractions is withdrawn from the top section as product fraction, at least one of the at least three side fractions is withdrawn from the top section as purge and at least one of the at least three side fractions is withdrawn from the second middle section of the middle dividing-wall distillation column as product fraction, wherein the purge is withdrawn during the method from the top section at a lower location than the product fraction being withdrawn from the top section of the middle dividing-wall distillation column.

Split-fatty acid crude material always contains in addition to the main fatty acid components certain amounts of impurity components, such as ketones, alkanes, odd-chained fatty acids. In order to get rid of these impurities, it is preferred that appropriate proportions of purge are drawn from the middle dividing-wall distillations as light end from the top and as middle purge between the main fatty acid product.

Good results are in particular obtained, when during the method in at least one and preferably each of the middle dividing-wall distillation columns the reflux ratio is adjusted to be between 1 and 70. For this purpose, as common, at least one and preferably each of the middle dividing-wall distillation columns has an overhead condenser being in fluid communication with the head and a reboiler being in fluid communication with the bottom of the middle dividing-wall distillation column. Likewise, preferably at least one and preferably each of the non-dividing-wall distillation columns has an overhead condenser being in fluid communication with the head and a reboiler being in fluid communication with the bottom of the non-dividing-wall distillation column.

For instance, the reflux ratio in the second middle dividing-wall column of the first variant of the embodiment of the present disclosure is preferably adjusted to be 1.5 to 3.0, such as about 2.3. Moreover, it is preferred that the reflux ratio in the second middle dividing-wall column of the second preferred embodiment of the present disclosure is adjusted to be 20 to 40, such as about 31.5, and that the reflux ratio in the third middle dividing-wall column of the second preferred embodiment is adjusted to be 3.5 to 7.0, such as about 5.1. In addition, it is preferred that the reflux ratio in the second middle dividing-wall column of the third preferred embodiment of the present disclosure is adjusted to be 20 to 40, such as about 31.5. that the reflux ratio in the third middle dividing-wall column of the third preferred embodiment is adjusted to be 3.5 to 7.0, such as about 5.1, and that the reflux ratio in the fifth middle dividing-wall column of the third preferred embodiment is adjusted to be 35 to 70, such as about 45.

In addition, it is preferred that during the method in at least one and preferably each of the middle dividing-wall distillation columns the pressure is adjusted to be between 0.5 and 10 KPa.

For instance, the pressure in the second middle dividing-wall column of the first variant of the embodiment of the present disclosure is preferably adjusted to be 1.0 to 2.0 KPa, such as about 1.5 KPa. Moreover, it is preferred that the pressure in the second middle dividing-wall column of the second preferred embodiment of the present disclosure is adjusted to be 2.5 to 5.0 KPa, such as about 4.0 KPa, and that the pressure in the third middle dividing-wall column of the second preferred embodiment is adjusted to be 0.5 to 1.5 KPa, such as about 0.8 KPa. In addition, it is preferred that the pressure in the second middle dividing-wall column of the third preferred embodiment of the present disclosure is adjusted to be 2.5 to 5.0 KPa, such as about 4.0 KPa, that the pressure in the third middle dividing-wall column of the third preferred embodiment is adjusted to be 0.5 to 1.5 KPa, such as about 0.8 KPa, and that the pressure in the fifth middle dividing-wall column of the third preferred embodiment is adjusted to be 5.0 to 10.0 KPa, such as about 8.0 KPa.

In principle, the method in accordance with the present disclosure is suitable to be used for fractionating any crude composition comprising at least six fatty acids having each a different chain length into at least four fractions. More preferably, the method in accordance with the present disclosure can be used for fractionating any crude composition comprising at least six fatty acids having each a different chain length into at least five fractions and even more preferably into at least six fractions. Most preferably, the method in accordance with the present disclosure can be used for fractionating any crude composition comprising at least seven fatty acids having each a different chain length into at least four fractions, more preferably at least five fractions, still more preferably at least six fractions and most preferably at least seven fractions

The method in accordance with the present disclosure is particularly suitable for fractionating palm kernel fatty acid and/or coconut fatty acid into five, six or preferably seven fractions. Therefore, it is preferred that the crude composition contains palm kernel fatty acid and/or coconut fatty acid and more preferred that the crude composition consists of palm kernel fatty acid and/or coconut fatty acid.

The method in accordance with the present disclosure allows in the aforementioned embodiments the palm kernel fatty acid and/or coconut fatty acid to be fractionated into four, five, six or seven fractions having an excellently high purity.

More specifically, the method allows withdrawal from the second middle dividing-wall distillation column of the first preferred embodiment, which comprises one middle dividing-wall distillation column, wherein this middle dividing-wall distillation column is the second distillation column, as side fraction—preferably from the top section—a C₁₂-fraction having a content of C₁₂-fatty acids of at least 98.0% by weight, whereas a C₁₄-fraction having a content of C₁₄-fatty acids of at least 98.0% by weight is withdrawn as side fraction—preferably from the second middle section—of the second middle dividing-wall distillation column, a C₁₆-fraction having a content of C₁₆-fatty acids of at least 98.0% by weight is withdrawn as side fraction of the third distillation column and a C₁₈ₚₗᵤₛ-fraction having a content of C₁₈-and longer fatty acids of at least 95.0% by weight is withdrawn as side fraction from the fourth distillation column. In addition, as side fraction from the first distillation column a combined C₈₋₁₀-fraction having a content of C₈₋₁₀-fatty acids of at least 98.0% by weight is withdrawn. More preferably, in this embodiment a C₁₂-fraction having a content of C₁₂-fatty acids of at least 99.0% by weight, more preferably of at least 99.5% by weight and most preferably of at least 99.9% by weight is withdrawn as side fraction from the second middle dividing-wall distillation column, whereas a C₁₈-fraction having a content of C₁₄-fatty acids of at least 99.0% by weight, more preferably of at least 99.5% by weight and most preferably of at least 99.9% by weight is withdrawn as side fraction—preferably from the second middle section—of the second middle dividing-wall distillation column, a C₁₆-fraction having a content of C₁₆-fatty acids of at least 99.0% by weight, more preferably of at least 99.5% by weight and most preferably of at least 99.8% by weight is withdrawn as side fraction of the third distillation column, a C₁₈ₚₗᵤₛ-fraction having a content of C₁₈-and longer fatty acids of at least 99.0% by weight, more preferably of at least 99.5% by weight and most preferably of at least 99.5% by weight is withdrawn as side fraction from the fourth distillation column and a combined C₈₋₁₀-fraction having a content of C₈₋₁₀-fatty acids of at least 98.0% by weight and more preferably of at least 99.0% by weight is withdrawn as side fraction from the first distillation column.

Furthermore, the method allows withdrawal from the second middle dividing-wall distillation column of the second preferred embodiment, which comprises the use of two middle dividing-wall distillation columns, wherein these middle dividing-wall distillation columns are the second and the third distillation columns, as side fraction—preferably from the top section—a C₁₀-fraction having a content of C₁₀-fatty acids of at least 98.0% by weight, whereas a C₁₂-fraction having a content of C₁₂-fatty acids of at least 98.0% by weight is withdrawn as side fraction—preferably from the second middle section—of the second middle dividing-wall distillation column, a C₁₄-fraction having a content of C₁₄-fatty acids of at least 98.0% by weight is withdrawn as side fraction—preferably from the top section—of the third middle dividing-wall distillation column, a C₁₆-fraction having a content of C₁₆-fatty acids of at least 98.0% is withdrawn as side fraction—preferably from the second section—of the third middle dividing-wall distillation column and a C₁₈ₚₗᵤₛ-fraction having a content of C₁₈-and longer fatty acids of at least 98.0% by weight is withdrawn as side fraction from the fourth distillation column. In addition, a C₈-fraction having a content of C₈-fatty acids of at least 98.0% may be withdrawn as side fraction from the first distillation column and a C₆-fraction having a content of C₆-fatty acids of at least 95.0% by weight may be withdrawn as overhead fraction from the first distillation column. More preferably, during the method of this embodiment a C₁₀-fraction having a content of C₁₀-fatty acids of at least 99.0% by weight, more preferably of at least 99.5% by weight and most preferably of at least 99.8% by weight is withdrawn as side fraction from the second middle dividing-wall distillation column, a C₁₂-fraction having a content of C₁₂-fatty acids of at least 99.0% by weight, more preferably of at least 99.5% by weight and most preferably of at least 99.9% by weight is withdrawn as side fraction from the second middle section of the second middle dividing-wall distillation column, a C₁₄-fraction having a content of C₁₄-fatty acids of at least 99.0% by weight, more preferably of at least 99.5% by weight and most preferably of at least 99.9% by weight is withdrawn as side fraction from the top section of the third middle dividing-wall distillation column, a C₁₆-fraction having a content of C₁₆-fatty acids of at least 99.0% by weight, more preferably of at least 99.5% by weight and most preferably of at least 99.8% by weight is withdrawn as side fraction from the second middle section of the third middle dividing-wall distillation column and a C₁₈ₚₗᵤₛ-fraction having a content of C₁₈- and longer fatty acids of at least 99.0% by weight. more preferably of at least 99.5% by weight and most preferably of at least 99.5% by weight is withdrawn as side fraction from the fourth distillation column. In addition, a C₆-fraction having a content of C₆-fatty acids of at least 95.0% and most preferably of at least 99.8% by weight may be withdrawn as overhead fraction from the first distillation column and a C₈-fraction having a content of C₈-fatty acids of at least 99.0% by weight, more preferably of at least 99.5% by weight and most preferably of at least 99.8% by weight may be withdrawn as overhead fraction from the first distillation column.

In the third preferred embodiment of the present disclosure, according to which the method the use of three middle dividing-wall distillation columns, wherein these middle dividing-wall distillation columns are the second, the third and the fifth distillation columns, as side fraction—preferably from the top section—from the second middle dividing-wall distillation column a C₁₀-fraction having a content of C₁₀-fatty acids of at least 98.0% by weight, more preferably of at least 99.0% by weight, even more preferably of at least 99.5% by weight and most preferably of at least 99.8% by weight is withdrawn, wherein a C₁₄-fraction having a content of C₁₂-fatty acids of at least 98.0% by weight, more preferably of at least 99.0% by weight, even more preferably of at least 99.5% by weight and most preferably of at least 99.9% by weight is withdrawn as side fraction—preferably from the second middle section—of the second middle dividing-wall distillation column, whereas from the third middle dividing-wall distillation column a C₁₄-fraction having a content of C₁₄-fatty acids of at least 98.0% by weight, more preferably of at least 99.0% by weight, even more preferably of at least 99.5% by weight and most preferably of at least 99.9% by weight is withdrawn as side fraction—preferably from the top section—and a C₁₆-fraction having a content of C₁₆-fatty acids of at least 98.0% by weight, more preferably of at least 99.0% by weight, even more preferably of at least 99.5% by weight and most preferably of at least 99.8% by weight is withdrawn as side fraction—preferably from the second middle section—of the third middle dividing-wall distillation column. From the fourth distillation column a C₁₈ₚₗᵤₛ-fraction having a content of C₁₈- and longer fatty acids of at least 98.0% by weight, more preferably of at least 99.0% by weight and most preferably of at least 99.5% by weight is withdrawn as side fraction. Finally, from the fifth middle dividing-wall distillation column a C₆-fraction having a content of C₆-fatty acids of at least 98.0% by weight, more preferably of at least 99.0% by weight, even more preferably of at least 99.5% by weight and most preferably of at least 99.8% by weight is withdrawn as side fraction—preferably from the top section—and a C₈-fraction having a content of C₈-fatty acids of at least 98.0% by weight, more preferably of at least 99.0% by weight, even more preferably of at least 99.5% by weight and most preferably of at least 99.8% by weight is withdrawn as side fraction—preferably from the second middle section—of the fifth middle dividing-wall distillation column.

In order to achieve a particular excellent separation efficiency with a particular low energy consumption, it is suggested in a further embodiment of the present disclosure that at least one and preferably all distillation columns comprise at least one element selected from the group consisting of trays, structured packings, random packings and arbitrary combinations of two or more of the aforementioned elements.

Good results are in particular obtained, when at least one and preferably all distillation columns comprise at least one or more structured packings.

Preferably, at least one of the middle dividing-wall distillation columns and more preferably all distillation columns, if more than one is used, each comprise i) two to four, more preferably two to three and most preferably two beds of structured packings in the top section. ii) two to four, more preferably two to three and most preferably two beds of structured packings in each of the middle sections and iii) one to three, more preferably one to two and most preferably one bed of structured packings in the bottom section.

It is further preferred that any of the structured packings is a cross-channel type structured packing, i.e. one which comprises a plurality of adjacent corrugated sheets, which are arranged parallel and in touching contact with each other so that an open space extending from one end to the opposite end of the at least two layers is provided between them so that a fluid may flow through it. The sheets may be metal sheets, which may be perforated by containing a plurality of opening or non-perforated. The sheets may be in particular metal gauze sheets, which may be perforated by containing a plurality of opening or non-perforated. The use of such cross-channel type structured packings, in particular those made of metal gauze sheets, allows to reduce the operating pressure at head of the distillation column and in particular middle dividing-wall distillation column such that the generation of low pressure steam to be withdrawn as purge stream is possible, but with keeping the bottom temperature at below 240° C.

Good results are in particular obtained, when any of the structured packings has a specific area of 60 to 750 m²/m³, more preferably of 120 to 500 m²/m³ and most preferably of 200 to 450 m²/m³.

A further aspect of the present disclosure is a plant for fractionating a crude composition comprising at least six fatty acids having each a different chain length into at least four fractions, in particular for performing a method of any of the preceding claims, wherein the plant comprises:

a first distillation column having a first inlet line for the crude composition, a first outlet line, which is connected with a first head and a second outlet line connected with a first bottom of the first distillation column,
a second distillation column having a second inlet line connected with the second outlet line, a third outlet line connected with a second head and a fourth outlet line connected with a second bottom of the second distillation column,
a third distillation having a third inlet line connected with the fourth outlet line, a fifth outlet line connected with a third head and a sixth outlet line connected with a third bottom of the third distillation column, and
a fourth distillation column having a fourth inlet line connected with the sixth outlet line, a seventh outlet line connected with a fourth head and an eighth outlet line connected with a fourth bottom of the fourth distillation column,

wherein at least one of the first distillation column, the second distillation column and the third distillation column is a middle dividing-wall column, which further comprises at least two outlet lines connected with a portion of the middle dividing-wall distillation column located between a head and a bottom of the middle dividing-wall distillation column.

In accordance with a variant of a first preferred embodiment, in which the plant comprises one middle dividing-wall distillation column, the plant comprises:

a first non-dividing-wall distillation column having an inlet line for the crude composition, an outlet line being connected with the head, an outlet line being connected with the bottom of the first non-dividing-wall distillation column and an outlet line being connected with a portion of the first non-dividing-wall distillation column being located between the head and the bottom of the first non-dividing-wall distillation column,
a second middle dividing-wall distillation column having an inlet line being connected with the outlet line, which is connected with the bottom of the first non-dividing-wall distillation column, having an outlet line being connected with the head, an outlet line being connected with the bottom of the second middle dividing-wall distillation column and at least two outlet lines each of which being connected with a portion of the second middle dividing-wall distillation column being located between the head and the bottom of the second middle dividing-wall distillation column,
a third non-dividing-wall distillation having an inlet line being connected with the outlet line, which is connected with the bottom of the second middle dividing-wall distillation column, having an outlet line being connected with the head, an outlet line being connected with the bottom of the third non-dividing-wall distillation column and an outlet line being connected with a portion of the third non-dividing-wall distillation column being located between the head and the bottom of the third non-dividing-wall distillation column, and
a fourth non-dividing-wall distillation column having an inlet line being connected with the outlet line, which is connected with the bottom of the third non-dividing-wall distillation column, having an outlet line being connected with the head, an outlet line being connected with the bottom of the fourth non-dividing-wall distillation column and an outlet line being connected with a portion of the fourth non-dividing-wall distillation column being located between the head and the bottom of the fourth non-dividing-wall distillation column.

In accordance with another variant of the first preferred embodiment, in which the plant comprises one middle dividing-wall distillation column, the plant comprises:

a first middle dividing-wall distillation column having an inlet line for the crude composition, an outlet line being connected with the head, an outlet line being connected with the bottom of the first middle dividing-wall distillation column and at least two outlet lines each of which being connected with a portion of the first middle dividing-wall distillation column being located between the head and the bottom of the first middle dividing-wall distillation column,
a second non-dividing-wall distillation column having an inlet line being connected with the outlet line, which is connected with the bottom of the first middle dividing-wall distillation column, having an outlet line being connected with the head, an outlet line being connected with the bottom of the second non-dividing-wall dividing-wall distillation column and one outlet line being connected with a portion of the second non-dividing-wall distillation column being located between the head and the bottom of the second non-dividing-wall distillation column,
a third non-dividing-wall distillation having an inlet line being connected with the outlet line, which is connected with the bottom of the second non-dividing-wall distillation column, having an outlet line being connected with the head, an outlet line being connected with the bottom of the third non-dividing-wall distillation column and an outlet line being connected with a portion of the third non-dividing-wall distillation column being located between the head and the bottom of the third non-dividing-wall distillation column, and
a fourth non-dividing-wall distillation column having an inlet line being connected with the outlet line, which is connected with the bottom of the third non-dividing-wall distillation column, having an outlet line being connected with the head, an outlet line being connected with the bottom of the fourth non-dividing-wall distillation column and an outlet line being connected with a portion of the fourth non-dividing-wall distillation column being located between the head and the bottom of the fourth non-dividing-wall distillation column.

In accordance with a second preferred embodiment, in which the plant comprises two middle dividing-wall distillation columns, the plant comprises:

a non-dividing-wall first distillation column having an inlet line for the crude composition, an outlet line being connected with the head, an outlet line being connected with the bottom of the first non-dividing-wall distillation column and an outlet line being connected with a portion of the first non-dividing-wall distillation column being located between the head and the bottom of the first non-dividing-wall distillation column,
a second middle dividing-wall distillation column having an inlet line being connected with the outlet line, which is connected with the bottom of the first non-dividing-wall distillation column, having an outlet line being connected with the head, an outlet line being connected with the bottom of the second middle dividing-wall distillation column and at least two outlet lines each of which being connected with a portion of the second middle dividing-wall distillation column being located between the head and the bottom of the second middle dividing-wall distillation column,
a third middle dividing-wall distillation column having an inlet line being connected with the outlet line, which is connected with the bottom of the second middle dividing-wall distillation column, having an outlet line being connected with the head, an outlet line being connected with the bottom of the third middle dividing-wall distillation column and at least two outlet lines each of which being connected with a portion of the third middle dividing-wall distillation column being located between the head and the bottom of the third middle dividing-wall distillation column, and
a fourth non-dividing-wall distillation column having an inlet line being connected with the outlet line, which is connected with the bottom of the third middle dividing-wall distillation column, having an outlet line being connected with the head, an outlet line being connected with the bottom of the fourth non-dividing-wall distillation column and an outlet line being connected with a portion of the fourth non-dividing-wall distillation column being located between the head and the bottom of the fourth non-dividing-wall distillation column.

In accordance with a third preferred embodiment, in which the plant comprises three middle dividing-wall distillation columns, the plant comprises in addition of the four distillation columns of the aforementioned second preferred embodiment a fifth middle dividing-wall distillation column having an inlet line being connected with the outlet line, which is connected with the head of the first distillation column, wherein the fifth middle dividing-wall distillation column has an outlet line being connected with the head, an outlet line being connected with the bottom and at least two outlet lines being connected with a portion of the middle dividing-wall distillation column being located between the head and the bottom of the fifth middle dividing-wall distillation column. Thus, the plant of this embodiment comprises:

a non-dividing-wall first distillation column having an inlet line for the crude composition, an outlet line being connected with the head, an outlet line being connected with the bottom of the first non-dividing-wall distillation column and an outlet line being connected with a portion of the first non-dividing-wall distillation column being located between the head and the bottom of the first non-dividing-wall distillation column,
a second middle dividing-wall distillation column having an inlet line being connected with the outlet line, which is connected with the bottom of the first non-dividing-wall distillation column, having an outlet line being connected with the head, an outlet line being connected with the bottom of the second middle dividing-wall distillation column and at least two outlet lines each of which being connected with a portion of the second middle dividing-wall distillation column being located between the head and the bottom of the second middle dividing-wall distillation column.
a third middle dividing-wall distillation column having an inlet line being connected with the outlet line, which is connected with the bottom of the second middle dividing-wall distillation column, having an outlet line being connected with the head, an outlet line being connected with the bottom of the third middle dividing-wall distillation column and at least two outlet lines each of which being connected with a portion of the third middle dividing-wall distillation column being located between the head and the bottom of the third middle dividing-wall distillation column,
a fourth non-dividing-wall distillation column having an inlet line being connected with the outlet line, which is connected with the bottom of the third middle dividing-wall distillation column, having an outlet line being connected with the head, an outlet line being connected with the bottom of the fourth non-dividing-wall distillation column and an outlet line being connected with a portion of the fourth non-dividing-wall distillation column being located between the head and the bottom of the fourth non-dividing-wall distillation column, and
a fifth middle dividing-wall distillation column having an inlet line being connected with the outlet line, which is connected with the head of the first distillation column, wherein the fifth middle dividing-wall distillation column has an outlet line being connected with the head, an outlet line being connected with the bottom and at least two outlet lines being connected with a portion of the middle dividing-wall distillation column being located between the head and the bottom of the fifth middle dividing-wall distillation column.

It is suggested in a further embodiment of the present disclosure that at least one and preferably all of the middle dividing-wall columns comprises at least three outlet lines being connected with a portion of the middle dividing-wall distillation column being located between the head and the bottom of the middle dividing-wall distillation column.

In accordance with a particularly preferred embodiment of the present disclosure, at least one and preferably each of the at least one middle dividing-wall column(s) comprises a dividing-wall (particularly preferably one, but not two or more dividing-walls) extending, seen from the bottom to the top of the middle dividing-wall distillation column, at least essentially vertically downwards so that the middle dividing-wall distillation column comprises above the dividing-wall a top section, below the dividing-wall a bottom section, on one side of the dividing-wall a first middle section and on the opposite side of the dividing-wall a second middle section, wherein essentially vertically downwards means that the angle between the dividing-wall and the length axis of the middle dividing-wall distillation column is at most 20°, preferably at most 10°, more preferably at most 5° and most preferably 0°. Preferably, the inlet line of at least one and preferably each of the middle dividing-wall distillation columns is connected with the first middle section of the middle dividing-wall distillation column, wherein at least one of the at least two outlet lines being connected with a portion of the middle dividing-wall distillation column being located between the head and the bottom of the middle dividing-wall distillation column is connected with the top section and at least one of the at least two outlet lines is connected with the second middle section of the middle dividing-wall distillation column.

In accordance with a further particularly preferred embodiment of the present disclosure, at least one and preferably all of the middle dividing-wall columns comprises at least three outlet lines being connected with a portion of the middle dividing-wall distillation column being located between the head and the bottom of the middle dividing-wall distillation column, wherein preferably the inlet line of at least one and preferably each of the middle dividing-wall distillation columns is connected with the first middle section of the middle dividing-wall distillation column, wherein at least two of the at least three outlet lines are connected with the top section and at least one of the at least three outlet lines is connected with the second middle section of the middle dividing-wall distillation column. Preferably one of the at least two outlet lines being connected with the top section and more preferably the lower one of the at least two outlet lines being connected with the top section functions as purge withdrawal line.

Preferably, at least one and, if more than one middle dividing-wall distillation column is present, more preferably each of the middle dividing-wall distillation columns comprise a dividing-wall extending, seen from the bottom to the top of the middle dividing-wall distillation column, from a point being located at 10 to 40% of the distance from the bottom to the top of the middle dividing-wall distillation column to a point being located at 50 to 90% of the distance from the bottom to the top of the middle dividing-wall distillation column at least essentially vertically downwards so that the middle dividing-wall distillation column comprises above the dividing-wall a top section functioning as refinement distillation section, below the dividing-wall a bottom section functioning as distillation section, on one side of the dividing-wall a first middle section functioning as feed section and on the opposite side of the dividing-wall a second middle section functioning as lateral extraction section. Again, essentially vertically downwards means in accordance with the present disclosure that the angle between the dividing-wall and the length axis of the first dividing-wall distillation column is at most 20°, preferably at most 10°, more preferably at most 5° and most preferably 0°. More preferably, at least one and still more preferably each of the middle dividing-wall distillation columns comprise a dividing-wall extending, seen from the bottom to the top of the middle dividing-wall distillation column, from a point being located at 20 to 40% of the distance from the bottom to the top of the middle dividing-wall distillation column to a point being located at 50 to 80% of the distance from the bottom to the top of the middle dividing-wall distillation column at least essentially vertically downwards Most preferably, at least one and still more preferably each of the middle dividing-wall distillation columns comprise a dividing-wall extending, seen from the bottom to the top of the middle dividing-wall distillation column, from a point being located at 25 to 35% of the distance from the bottom to the top of the middle dividing-wall distillation column to a point being located at 55 to 75% of the distance from the bottom to the top of the middle dividing-wall distillation column at least essentially vertically downwards.

Good results in terms of high separation efficiency at low energy consumption are in particular obtained, when in at least one and preferably each of the middle dividing-wall distillation columns the dividing-wall extends over 20 to 80%, more preferably over 30 to 70% and most preferably over 30 to 60% of the height of the middle dividing-wall distillation column. The height of the middle dividing-wall distillation column is defined in this connection as the straight distance between the top and the bottom of the respective middle dividing-wall distillation column.

As set out above, in a further embodiment of the present disclosure, the inlet line of at least one and preferably each of the middle dividing-wall distillation columns is connected with the first middle section of the respective middle dividing-wall distillation column, wherein preferably, in at least one and preferably each of the middle dividing-wall distillation columns one of the at least two outlet lines each of which being connected with a portion of the middle dividing-wall distillation column being located between the head and the bottom of the second middle dividing-wall distillation column is connected with the top section and another one of these at least two outlet lines is connected with the second middle section of the respective middle dividing-wall distillation column.

As mentioned above, it is further preferred that at least one of the at least two outlet lines being connected with a portion of the middle dividing-wall distillation column being located between the head and the bottom of the middle dividing-wall distillation column and more preferably at least one of the at least two outlet lines being connected with the top section of the middle dividing-wall distillation column functions as purge withdrawal line.

In accordance with another preferred embodiment of the present disclosure, at least one and preferably all distillation columns of the plant comprise at least one element selected from the group consisting of trays, structured packings, random packings and arbitrary combinations of two or more of the aforementioned elements.

Preferably, at least one and more preferably all distillation columns comprise at least one or more structured packings.

Good results are in particular obtained, when one and, if more than one middle dividing-wall distillation column is present, all middle dividing-wall distillation columns each comprise i) two to four, preferably two to three and more preferably two beds of structured packings in the top section, ii) two to four, preferably two to three and more preferably two beds of structured packings in each of the middle sections and iii) one to three, preferably one to two and more preferably one bed of structured packings in the bottom section.

In a further embodiment of the present disclosure, it is suggested that the structured packing is a cross-channel type structured packing which comprises a plurality of adjacent corrugated sheets, which are arranged parallel and in touching contact with each other so that an open space extending from one end to the opposite end of the at least two layers is provided between them so that a fluid may flow through it. The sheets may be metal sheets, which may be perforated by containing a plurality of opening or non-perforated. The sheets may be in particular metal gauze sheets, which may be perforated by containing a plurality of opening or non-perforated.

Good results are in particular obtained, when any of the structured packings has a specific area of 60 to 750 m²/m³, more preferably of 120 to 500 m²/m³ and most preferably of 200 to 450 m²/m³.

### BRIEF DESCRIPTION OF THE DRAWINGS

FIG. 1 illustrates a schematic view of a conventional plant for fractionating palm kernel fatty acid.

FIG. 2 illustrates a schematic view of a plant for fractionating palm kernel fatty acid in accordance with one embodiment of the present disclosure.

FIG. 3 illustrates a schematic view of a plant for fractionating palm kernel fatty acid in accordance with another embodiment of the present disclosure.

FIG. 4 illustrates a schematic view of a plant for fractionating palm kernel fatty acid in accordance with an embodiment of the present disclosure.

### DETAILED DESCRIPTION

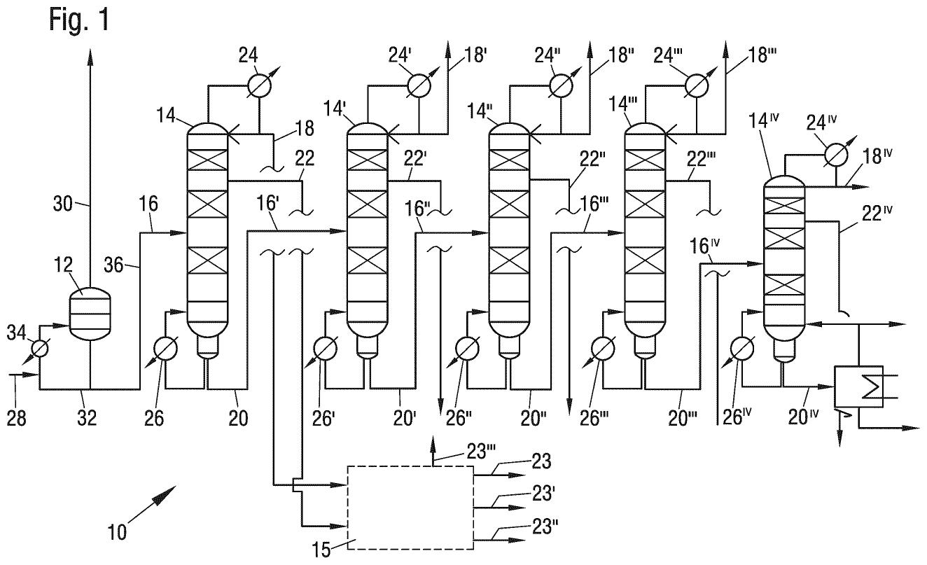

FIG. 1 illustrates schematically a conventional plant 1 for fractionating palm kernel fatty acid. The plant 1 comprises, from its upstream to its downstream end, a drier or deaerator 12, respectively, five distillation column 14 to 14^{iv} and a multipurpose fractionation unit 15 comprising two further distillation columns (not shown). Each of the five distillation columns 14 to 14 comprises an inlet line 16 to 16″, an outlet line 18 to 18^{iv} being connected with the head of the respective distillation column 14 to 14^{iv}, an outlet line 20 to 20^{iv} being connected with the bottom of the respective distillation column 14 to 14^{iv} and an outlet 22 to 22^{iv} being connected with a portion of the respective distillation column 14 to 14^{iv} being located between its head and its bottom. Each of the inlet lines 16′ to 16^{iv} of the second to fifth distillation column 14′ to 14^{iv} is connected with the outlet line 20 to 20ⁱⁱⁱ being connected with the bottom of the adjacent upstream distillation column 14 to 14ⁱⁱⁱ. The outlet lines 18 and 22 of the first distillation column 14 are connected with the multipurpose fractionation unit 15, which has four outlet lines 23, 23′, 23″, 23″′. Each of the distillation columns comprises a condenser 24 to 24^{iv} being in fluid communication with the head and a reboiler 26 to 26″ being in fluid communication with the bottom of the non-dividing-wall distillation column 14 to 14^{iv}. The drier or deaerator 12, respectively, is connected with a feed inlet line 28, a gas outlet line 30, a recirculation line 32 comprising a reboiler 34 and an outlet line 36, which is connected with the inlet line 16 to the first distillation column 14.

During the operation of this plant 1, crude palm kernel fatty acid is continuously fed via the feed inlet line 28 and the recirculation line 32 into the drier or deaerator 12, respectively. Gas separated within the drier or deaerator 12, respectively, is withdrawn from the drier or deaerator 12, respectively, via the gas outlet line 30, whereas the dried and deaerated crude palm kernel fatty acid is fed via the inlet line 16 into the first distillation column 14. The crude palm kernel fatty acid is distilled in the first distillation column 14 so as to obtain an overhead fraction as C₆-fraction of the palm kernel fatty acid, which is withdrawn from the first distillation column 14 via the outlet line 18, a side fraction as C₈₋₁₀-fraction of the palm kernel fatty acid, which is withdrawn from the first distillation column 14 via the outlet line 22, and a bottom fraction with the remaining fatty acids, which is withdrawn from the first distillation column 14 via the outlet line 20. The C₆-fraction and the C₈₋₁₀-fraction of the palm kernel fatty acid are fed via lines 18 and 22 into the multipurpose fractionation unit 15, in which these fractions are fractionated into a C₆-fraction, a C₈-fraction and a C₁₀-fraction, which are withdrawn via the outlet lines 23, 23′, 23″. Moreover, a purge stream is withdrawn from the multipurpose fractionation unit 15 via the outlet line 23′″. The bottom fraction of the first distillation column 14 is fed via lines 20, 16′ into the second distillation column 14′, in which it is distilled so as to obtain a light end as overhead fraction, which is withdrawn from the second distillation column 14′ via the outlet line 18′, a side fraction as C₁₂-fraction of the palm kernel fatty acid, which is withdrawn from the second distillation column 14′ via the outlet line 22′, and a bottom fraction with the remaining fatty acids, which is withdrawn from the second distillation column 14″ via the outlet line 20′. The bottom fraction of the second distillation column 14′ is fed via lines 20′, 16″ into the third distillation column 14″, in which it is distilled so as to obtain a light end as overhead fraction, which is withdrawn from the third distillation column 14″ via the outlet line 18″, a side fraction as C₁₄-fraction of the palm kernel fatty acid. which is withdrawn from the third distillation column 14″ via the outlet line 22″, and a bottom fraction with the remaining fatty acids, which is withdrawn from the third distillation column 14″ via the outlet line 20″. Likewise, the bottom fraction of the third distillation column 14″ is fed via lines 20″, 16″′ into the fourth distillation column 14″′, in which it is distilled so as to obtain a light end as overhead fraction, which is withdrawn from the fourth distillation column 14″′ via the outlet line 18″′, a side fraction as C₁₆-fraction of the palm kernel fatty acid. which is withdrawn from the fourth distillation column 14″′ via the outlet line 22″′, and a bottom fraction with the remaining fatty acids, which is withdrawn from the fourth distillation column 14″′ via the outlet line 20″′. Finally, the bottom fraction of the fourth distillation column 14″′ is fed via lines 20″′, 16^{iv} into the fifth distillation column 14^{iv}, in which it is distilled so as to obtain a light end as overhead fraction, which is withdrawn from the fourth distillation column 14^{iv} via the outlet line 18^{iv}, a side fraction as C₁₈ₚₗᵤₛ-fraction of the palm kernel fatty acid, which is withdrawn from the fifth distillation column 14^{iv} via the outlet line 22^{iv}, and a bottom fraction with the residuals.

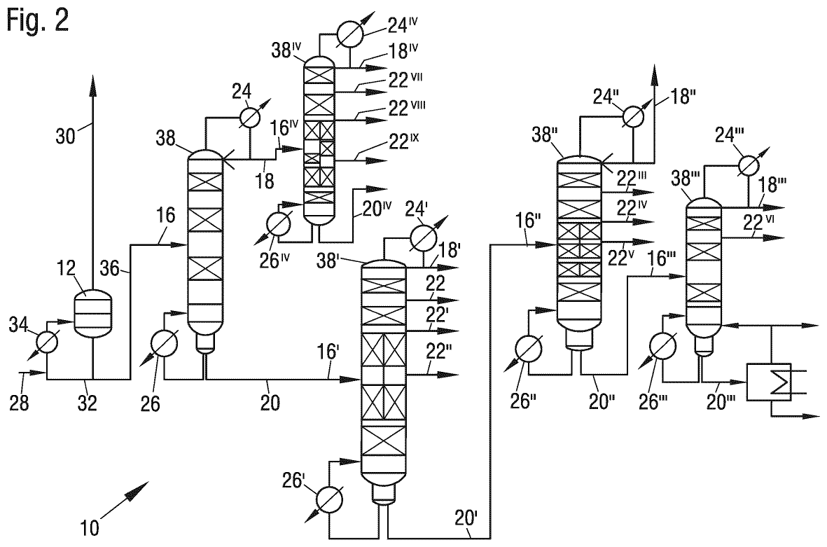

FIG. 2 illustrates schematically a plant 10 for fractionating palm kernel fatty acid in accordance with one embodiment of the present disclosure, which corresponds to the third preferred embodiment as described above. The plant 10 comprises a drier or deaerator 12, respectively, a first distillation column 38 embodied as non-dividing-wall column, a second distillation column 38′ embodied as middle dividing-wall column, a third distillation column 38″ embodied as middle dividing-wall column, a fourth distillation column 38′″ embodied as non-dividing-wall column as well as a fifth distillation column 38^{iv} embodied as middle dividing-wall column. The first distillation column 38 has an inlet line 16 for the crude composition, an outlet line 18 being connected with the head and an outlet line 20 being connected with the bottom of the first distillation column 38. The inlet line 16 of the first distillation column 38 is connected with the outlet line 36 of the drier or deaerator 12, respectively, which is embodied as in FIG. 1, i.e, which is connected with a feed inlet line 28, a gas outlet line 30, a recirculation line 32 comprising a reboiler 34 and the outlet line 36. The second middle dividing-wall distillation column 38′ has an inlet line 16′ being connected with the outlet line 20, which is connected with the bottom of the first distillation column 38, wherein the second middle dividing-wall distillation column 38′ has an outlet line 18″ being connected with the head, an outlet line 20′ being connected with the bottom and three outlet lines 22, 22′, 22″ each of which being connected with a portion of the middle dividing-wall distillation column being located between the head and the bottom of the second middle dividing-wall distillation column 38′. Likewise, the third middle dividing-wall distillation 38″ has an inlet line 16″ being connected with the outlet line 20′, which is connected with the bottom of the second middle dividing-wall distillation column 38′, wherein the third middle dividing-wall distillation 38″ has an outlet line 18″ being connected with the head, an outlet line 20″ being connected with the bottom and three outlet lines 22″′, 22^{iv}, 22^{v} each of which being connected with a portion of the third middle dividing-wall distillation column 38″ being located between the head and the bottom of the third middle dividing-wall distillation column 38″. The fourth distillation column 38″′ has an inlet line 16′″ being connected with the outlet line 20″, which is connected with the bottom of the third middle dividing-wall distillation column 38″, wherein the fourth distillation column 38″ has an outlet line 18′″ being connected with the head, an outlet line 20″′ being connected with the bottom and an outlet line 22^{vi} being connected with a portion of the fourth distillation column 38″ being located between the head and the bottom of the fourth distillation column 38″. Finally, the fifth middle dividing-wall distillation 38^{iv} has an inlet line 16″ being connected with the outlet line 18, which is connected with the head of the first dividing-wall distillation column 38, wherein the fifth middle dividing-wall distillation 38^{iv} has an outlet line 18^{iv} being connected with the head, an outlet line 20″ being connected with the bottom and three outlet lines 22^{vii}, 22^{vii}, 22^{viii} each of which being connected with a portion of the fifth middle dividing-wall distillation column 38^{iv} being located between the head and the bottom of the fifth middle dividing-wall distillation column 38^{iv}.

During the operation of this plant 10, crude palm kernel fatty acid is continuously fed via the feed inlet line 28 and the recirculation line 32 into the drier or deaerator 12, respectively. Gas separated within the drier or deaerator 12, respectively, is withdrawn from the drier or deaerator 12, respectively, via the gas outlet line 30, whereas the dried and deaerated crude palm kernel fatty acid is fed via the inlet line 16 into the first distillation column 38. The crude palm kernel fatty acid is distilled in the first distillation column 38 so as to obtain an overhead fraction including C₆₋₈-fatty acids, which is withdrawn from the first distillation column 38 via the outlet line 18, and a bottom fraction with the remaining fatty acids, which is withdrawn from the first distillation column 38 via the outlet line 20. The bottom fraction of the first distillation column 38 is fed via lines 20, 16′ into the second middle dividing-wall distillation column 38′, in which it is distilled so as to obtain a light end as overhead fraction, which is withdrawn from the second middle dividing-wall distillation column 38′ via the outlet line 18′, a side fraction as C₁₀-fraction of the palm kernel fatty acid, which is withdrawn from the second middle dividing-wall distillation column 38′ via the outlet line 22, a purge stream, which is withdrawn from the second middle dividing-wall distillation column 38′ via the outlet line 22″, a side fraction as C₁₂-fraction of the palm kernel fatty acid, which is withdrawn from the second middle dividing-wall distillation column 38′ via the outlet line 22″, and a bottom fraction with the remaining fatty acids, which is withdrawn from the second middle dividing-wall distillation column 38′ via the outlet line 20′. The bottom fraction of the second distillation column 38′ is fed via lines 20′, 16″ into the third middle dividing-wall distillation column 38″, in which it is distilled so as to obtain a light end as overhead fraction, which is withdrawn from the third middle dividing-wall distillation column 38″ via the outlet line 18″, a side fraction as C₁₄-fraction of the palm kernel fatty acid, which is withdrawn from the third middle dividing-wall distillation column 38″ via the outlet line 22″′, a purge stream, which is withdrawn from the third middle dividing-wall distillation column 38″ via the outlet line 221, a side fraction as C₁₆-fraction of the palm kernel fatty acid, which is withdrawn from the third middle dividing-wall distillation column 38″ via the outlet line 22″, and a bottom fraction with the remaining fatty acids, which is withdrawn from the third middle dividing-wall distillation column 38″ via the outlet line 20″. The bottom fraction of the third distillation column 38″ is fed via lines 20″, 16″′ into the fourth distillation column 38″′, in which it is distilled so as to obtain a light end as overhead fraction, which is withdrawn from the fourth distillation column 38″′ via the outlet line 18″′, a side fraction as C₁₈ₚₗᵤₛ-fraction of the palm kernel fatty acid, which is withdrawn from the fourth distillation column 38″′ via the outlet line 22^{vi}, and a bottom fraction, which is withdrawn from the fourth distillation column 38″′ via the outlet line 20″′. Finally, the head fraction of the first distillation column 38 is fed via lines 18, 16^{iv} into the fifth middle dividing-wall distillation column 38^{iv}, in which it is distilled so as to obtain a light end as overhead fraction, which is withdrawn from the fifth middle dividing-wall distillation column 38^{iv} via the outlet line 18^{iv}, a side fraction as C₆-fraction of the palm kernel fatty acid, which is withdrawn from the fifth middle dividing-wall distillation column 38″ via the outlet line 22^{vii}, a purge stream, which is withdrawn from the fifth middle dividing-wall distillation column 38^{iv} via the outlet line 22^{viii}, a side fraction as C₈-fraction of the palm kernel fatty acid, which is withdrawn from the fifth middle dividing-wall distillation column 38^{iv} via the outlet line 22^{vix}, and a purge as bottom fraction, which is withdrawn from the fifth middle dividing-wall distillation column 381^{iv} via the outlet line 20^{iv}.

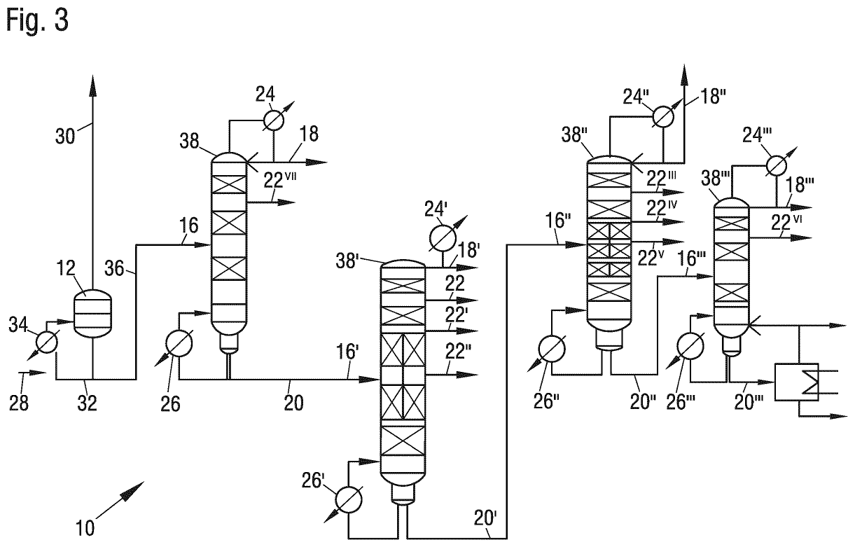

FIG. 3 illustrates schematically a plant 10 for fractionating palm kernel fatty acid in accordance with another embodiment of the present disclosure, which corresponds to the second preferred embodiment as described above. The plant 10 corresponds to that of FIG. 2 except that the fifth distillation column 38^{iv} is absent. Instead, the first distillation column 18 comprises in addition to the outlet line 18 of the head and the outlet line 20 of the bottom an outlet line 22″′ of the side of the first distillation column 18.

The operation performed during the operation of this plant 10 is the same as that performed during the operation of the plant 10 shown in FIG. 2 except that the head product of the first distillation column 18 withdrawn via line 18 is a C₆-fraction of the palm kernel fatty acid having a purity of about 95% and furthermore from the side of the first distillation column 18 via line 22^{vii} a C₆-fraction of the palm kernel fatty acid having a purity of about 99.5% is withdrawn.

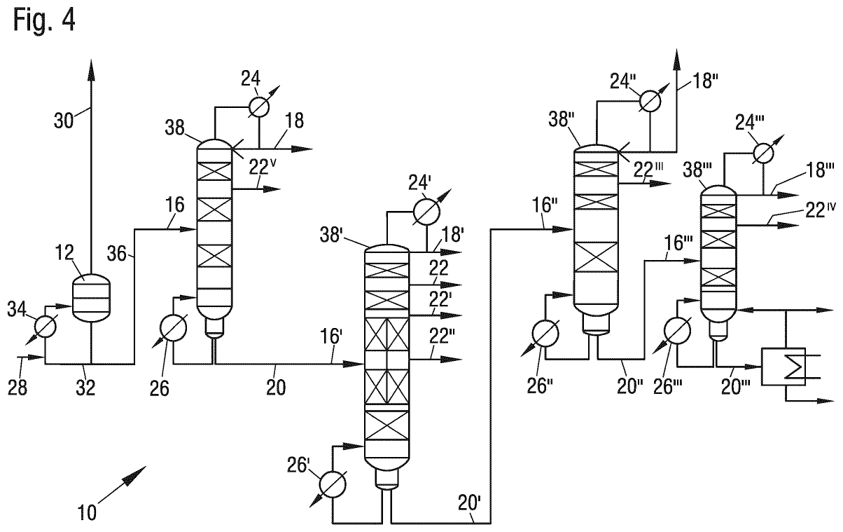

FIG. 4 illustrates schematically a plant 10 for fractionating palm kernel fatty acid in accordance with another embodiment of the present disclosure, which corresponds to the first preferred embodiment as described above. The plant 10 corresponds to that of FIG. 3 except that the third distillation column 38″is not a middle dividing-wall column, but a non-dividing-wall column.

During the operation of this plant 10, crude palm kernel fatty acid is continuously fed via the feed inlet line 28 and the recirculation line 32 into the drier or deaerator 12, respectively. Gas separated within the drier or deaerator 12, respectively, is withdrawn from the drier or deaerator 12, respectively, via the gas outlet line 30, whereas the dried and deaerated crude palm kernel fatty acid is fed via the inlet line 16 into the first distillation column 38. The crude palm kernel fatty acid is distilled in the first distillation column 38 so as to obtain an overhead fraction including lights, which is withdrawn from the first distillation column 38 via the outlet line 18, a C₈₋₁₀-fatty acids fraction withdrawn via the outlet line 22^{v} and a bottom fraction with the remaining fatty acids, which is withdrawn from the first distillation column 38 via the outlet line 20. The bottom fraction of the first distillation column 38 is fed via lines 20, 16′ into the second middle dividing-wall distillation column 38′, in which it is distilled so as to obtain a light end as overhead fraction, which is withdrawn from the second middle dividing-wall distillation column 38′ via the outlet line 18′, a side fraction as C₁₂-fraction of the palm kernel fatty acid, which is withdrawn from the second middle dividing-wall distillation column 38′ via the outlet line 22, a purge stream, which is withdrawn from the second middle dividing-wall distillation column 38′ via the outlet line 22′, a side fraction as C₁₈-fraction of the palm kernel fatty acid, which is withdrawn from the second middle dividing-wall distillation column 38′ via the outlet line 22″, and a bottom fraction with the remaining fatty acids, which is withdrawn from the second middle dividing-wall distillation column 38′ via the outlet line 20′. The bottom fraction of the second distillation column 38′ is fed via lines 20′, 16″ into the third non-dividing-wall distillation column 38″, in which it is distilled so as to obtain a light end as overhead fraction, which is withdrawn from the third non-dividing-wall distillation column 38″ via the outlet line 18″, a side fraction as C₁₆-fraction of the palm kernel fatty acid, which is withdrawn from the third non-dividing-wall distillation column 38″ via the outlet line 22′″ and a bottom fraction with the remaining fatty acids, which is withdrawn from the third non-dividing-wall distillation column 38″ via the outlet line 20″. The bottom fraction of the third distillation column 38″ is fed via lines 20″, 16″′ into the fourth distillation column 38″′, in which it is distilled so as to obtain a light end as overhead fraction, which is withdrawn from the fourth distillation column 38″′ via the outlet line 18′″, a side fraction as C₁₈ₚₗᵤₛ-fraction of the palm kernel fatty acid, which is withdrawn from the fourth distillation column 38′″ via the outlet line 22^{iv}, and a bottom fraction which is withdrawn from the fourth distillation column 38″′ via the outlet line 20′″.

1. A method for fractionating a crude composition comprising at least six fatty acids each having a different chain length into at least four fractions, the method comprising:
feeding the crude composition into a first distillation column and distilling it so as to obtain at least a first overhead fraction and a first bottom fraction;
feeding the first bottom fraction of the first distillation column to a second distillation column and distilling it so as to obtain at least a second overhead fraction and a second bottom fraction;
feeding the second bottom fraction of the second distillation column to a third distillation column and distilling it so as to obtain at least a third overhead fraction and a third bottom fraction; and
feeding the third bottom fraction of the third distillation column to a fourth distillation column and distilling it so as to obtain at least a fourth overhead fraction, a fourth side fraction and a fourth bottom fraction,
wherein at least one of the first distillation column, the second distillation column and the third distillation column is a middle dividing-wall column, from which also at least two side fractions are obtained.

2. The method of claim 1, which comprises the following steps: wherein
the first distillation column is a first non-dividing-wall distillation column and the crude composition is distilled so as to obtain at least the first overhead fraction, a first side fraction and the first bottom fraction,
the second distillation column is a second middle dividing-wall distillation column and the first bottom fraction of the first non-dividing-wall distillation column is distilled so as to obtain at least the first overhead fraction, at least two second side fractions and the second bottom fraction,
the third distillation column is a third non-dividing-wall distillation column and the second bottom fraction of the second middle dividing-wall distillation column is distilled so as to obtain at least the third overhead fraction, a third side fraction and the third bottom fraction, and
feeding the bottom fraction of the third non dividing-wall distillation column to the fourth distillation column is a fourth non-dividing-wall distillation column and the third bottom fraction of the third non-dividing-wall distillation column is distilled so as to obtain at least the fourth overhead fraction, the fourth side fraction and the fourth bottom fraction.

3. The method of claim 1, wherein
the first distillation column is a first middle dividing-wall distillation column and the crude composition is distilled so as to obtain at least the first overhead fraction, at least two first side fractions and the first bottom fraction,
the second distillation column is a second non-dividing-wall distillation column and the first bottom fraction of the first middle dividing-wall column is distilled so as to obtain at least the second overhead fraction, a second side fraction and the second bottom fraction,
the third distillation column is a third non-dividing-wall distillation column and the second bottom fraction of the second non-dividing-wall distillation column is distilled so as to obtain at least the third overhead fraction, a third side fraction and the third bottom fraction, and
the fourth distillation column is a fourth non-dividing-wall distillation column and the third bottom fraction of the third non-dividing-wall distillation column is distilled so as to obtain at least the fourth overhead fraction, the fourth side fraction and the fourth bottom fraction.

4. The method of claim 1, wherein
the first distillation column is a first non-dividing-wall distillation column and the crude composition is distilled so as to obtain at least the first overhead fraction, a first side fraction and the first bottom fraction,
the second distillation column is a second middle dividing-wall distillation column and the first bottom fraction of the first non-dividing-wall distillation column is distilled so as to obtain at least the second overhead fraction, at least two second side fractions and the second bottom fraction.
the third distillation column is a third middle dividing-wall distillation column and the second bottom fraction of the second middle dividing-wall distillation column is distilled so as to obtain at least the third overhead fraction, at least two third side fractions and the third bottom fraction, and
the fourth distillation column is a fourth non-dividing-wall distillation column and the third bottom fraction of the third middle dividing-wall distillation column is distilled so as to obtain at least the fourth overhead fraction, the fourth side fraction and the fourth bottom fraction.

5. The method of claim 4, further comprising:
feeding the first overhead fraction of the first non-dividing-wall distillation column to a fifth middle dividing-wall distillation column and distilling it so as to obtain a fifth overhead fraction, at least two fifth side fractions and a fifth bottom fraction.

6. The method of claim 1, wherein
at least one of the first distillation column, the second distillation column, the third distillation column and the fourth distillation column is a middle dividing-wall column from which at least three side fractions are obtained.

7. The method of claim 1, wherein
at least one of the first distillation column, the second distillation column, the third distillation column and the fourth distillation column is a middle dividing-wall distillation column comprising a dividing-wall extending, seen from a bottom to a top of the middle dividing-wall distillation column, at least essentially vertically downwards so that the middle dividing-wall distillation column comprises above the dividing-wall a top section, below the dividing-wall a bottom section, on one side of the dividing-wall a first middle section and on an opposite side of the dividing-wall a second middle section, wherein essentially vertically downwards means that an angle between the dividing-wall and a length axis of the middle dividing-wall distillation column is at most 20°, wherein a feed of the middle dividing-wall distillation column is fed into the first middle section of the middle dividing-wall distillation column, wherein at least one of the at least two side fractions is withdrawn from the top section and at least one of the at least two side fractions is withdrawn from the second middle section of the middle dividing-wall distillation column.

8. The method of claim 7, wherein
from the middle dividing-wall distillation column at least three side fractions are obtained, wherein from the middle dividing-wall distillation column at least two of the at least three side fractions are withdrawn from the top section and at least one of the at least three side fractions is withdrawn from the second middle section of the middle dividing-wall distillation column.

9. The method of claim 1, wherein
at least one of the of the first distillation column, the second distillation column, the third distillation column and the fourth distillation column is a middle dividing-wall distillation column comprising a dividing-wall extending, seen from a bottom to a top of the middle dividing-wall distillation column, from a point being located at 10 to 40% of a distance from the bottom to the top of the middle dividing-wall distillation column to a point being located at 50 to 90% of the distance from the bottom to the top of the middle dividing-wall distillation column at least essentially vertically downwards, and, wherein in the middle dividing-wall distillation column the dividing-wall extends over 20 to 80% of a height of the middle dividing-wall distillation column, wherein the height of the middle dividing-wall distillation column is a straight distance between the top and the bottom of the middle dividing-wall distillation column.

10. The method of claim 7, wherein
from the middle dividing-wall distillation column a purge is withdrawn from the top section of the middle dividing-wall distillation column.

11. The method of claim 1, wherein
the crude composition contains at least one of palm kernel fatty acid and coconut fatty acid.

12. The method of claim 4, wherein
from the second middle dividing-wall distillation column a C₁₀-fraction having a content of C₁₀-fatty acids of at least 98.0% by weight is withdrawn as a C₁₀-side fraction from a second top section and a C₁₂-fraction having a content of C₁₂-fatty acids of at least 98.0% by weight is withdrawn as a C₁₂-side fraction from a second middle section of the second middle dividing-wall distillation column, wherein from the third middle dividing-wall distillation column a C₁₄-fraction having a content of C₁₄-fatty acids of at least 98.0% by weight is withdrawn as a C₁₄-side fraction from a third top section and a C₁₆-fraction having a content of C₁₆-fatty acids of at least 98.0% by weight is withdrawn as a C₁₆-side fraction from a second middle section of the third middle dividing-wall distillation column, and wherein from the fourth distillation column a C₁₈ₚₗᵤₛ-fraction having a content of C₁₈-and longer fatty acids of at least 98.0% by weight is withdrawn as a C₁₈ₚₗᵤₛ-side fraction.

13. The method of claim 7, wherein
the middle dividing-wall distillation column comprises two to four beds of structured packings in the top section, two to four beds of structured packings in each of the middle sections and one to three beds of structured packings in the bottom section.

14. A plant for fractionating a crude composition into at least four fractions, the crude composition comprising at least six fatty acids having each a different chain length, the plant comprising:
a first distillation column having a first inlet line for the crude composition, a first outlet line connected with a first head and a second outlet line connected with a first bottom of the first distillation column.
a second distillation column having a second inlet line connected with the second outlet line, a third outlet line connected with a second head and a fourth outlet line connected with a second bottom of the second distillation column,
a third distillation having a third inlet line connected with the fourth outlet line, a fifth outlet line connected with a third head and a sixth outlet line connected with a third bottom of the third distillation column, and
a fourth distillation column having a fourth inlet line connected with the sixth outlet line, which is connected with the bottom of the third distillation-column; a seventh outlet line connected with a fourth head and an eighth outlet line connected with a fourth bottom of the fourth distillation column.
wherein at least one of the first distillation column, the second distillation column and the third distillation column is a middle dividing-wall column, which further comprises at least two outlet lines connected with a portion of the middle dividing-wall distillation column being located between a head and a bottom of the middle dividing-wall distillation column.

15. The plant of claim 14, wherein
the first distillation column is a first non-dividing-wall distillation column having a ninth outlet line connected with a portion of the first non-dividing-wall distillation column located between the head and the bottom of the first non dividing-wall-distillation column.
the second distillation column is a second middle dividing-wall distillation column having at least two tenth outlet lines each connected with a portion of the second middle dividing-wall distillation column located between the head and the bottom of the second distillation column,
the third distillation column is a third non-dividing-wall distillation having an eleventh outlet line being connected with a portion of the third non-dividing-wall distillation column located between the head and the bottom of the third distillation column, and
the fourth distillation column is a fourth non-dividing-wall distillation column having a twelfth outlet line connected with a portion of the fourth non-dividing-wall distillation column located between the head and the bottom of the fourth non dividing wall distillation column.

16. The plant of claim 14, which comprises: wherein
the first distillation column is a first middle dividing-wall distillation column having at least two thirteenth outlet lines each connected with a portion of the first middle dividing-wall distillation column located between the head and the bottom of the first distillation column.
the second distillation column is a second non-dividing-wall distillation column having a fourteenth outlet line connected with a portion of the second non-dividing-wall distillation column located between the head and the bottom of the second distillation column.
the third distillation column is a third non-dividing-wall distillation having a fifteenth outlet line connected with a portion of the third non-dividing-wall distillation column located between the head and the bottom of the third distillation column, and
the fourth distillation column is a fourth non-dividing-wall distillation column having a sixteenth outlet line connected with a portion of the fourth non-dividing-wall distillation column located between the head and the bottom of the fourth distillation column.

17. The plant of claim 14, wherein
the first distillation column is a first non-dividing-wall distillation column having a seventeenth outlet line connected with a portion of the first non-dividing-wall distillation column located between the head and the bottom of the first distillation column.
the second distillation column is a second middle dividing-wall distillation column having at least two eighteenth outlet lines each connected with a portion of the second middle dividing-wall distillation column located between the head and the bottom of the second distillation column,
the third distillation column is a third middle dividing-wall distillation column having at least two nineteenth outlet lines each connected with a portion of the third middle dividing-wall distillation column located between the head and the bottom of the third distillation column, and
the fourth distillation column is a fourth non-dividing-wall distillation column having a twentieth outlet line connected with a portion of the fourth non-dividing-wall distillation column located between the head and the bottom of the fourth distillation column.

18. The plant of claim 17, further comprising:
a fifth middle dividing-wall distillation column having a fifth inlet line connected with the first outlet line of the first distillation column, wherein the fifth middle dividing-wall distillation column has a twenty-first outlet line connected with a head, a twenty-second outlet line connected with a bottom and at least two twenty-third outlet lines connected with a portion of the fifth middle dividing-wall distillation column located between the head and the bottom of the fifth middle dividing-wall distillation column.

19. The plant of claim 17, wherein
at least one of the middle dividing-wall columns comprises at least three outlet lines connected with a portion of the at least one middle dividing-wall distillation column located between a head and a bottom of the at least one middle dividing-wall distillation column.

20. The plant of claim 19, wherein
the at least one middle dividing-wall column comprises a dividing-wall extending, seen from the bottom to a top of the at least one middle dividing-wall distillation column, at least essentially vertically downwards so that the at least one middle dividing-wall distillation column comprises above the dividing-wall a top section, below the dividing-wall a bottom section, on one side of the dividing-wall a first middle section and on an opposite side of the dividing-wall a second middle section, wherein essentially vertically downwards means that an angle between the dividing-wall and a length axis of the middle dividing-wall distillation column is at most 20°, wherein an inlet line of the at least one middle dividing-wall distillation columns is connected with the first middle section of the middle dividing-wall distillation column. wherein at least one of the at least three outlet lines connected with a portion of the middle dividing-wall distillation column located between the head and the bottom of the at least one middle dividing-wall distillation column is connected with the top section and at least one of the at least three outlet lines is connected with the second middle section of the at least one middle dividing-wall distillation column.

21. The plant of claim 20, wherein
a sixth inlet line of the at least one middle dividing-wall distillation column is connected with the first middle section of the middle dividing-wall distillation column, and at least two of the at least three outlet lines are connected with the top section and at least one of the at least three outlet lines is connected with the second middle section of the middle dividing-wall distillation column.
